(19) United States
(12) Patent Application Publication
(10) Pub. No.: US 2003/0139663 A1
(43) Pub. Date: Jul. 24, 2003

(54) Registration procedure in projective intra-operative 3D imaging

(75) Inventor: Rainer Graumann, Hoechstadt (DE)

Correspondence Address: SCHIFF HARDIN & WAITE, 6600 SEARS TOWER, 233 S WACKER DR, CHICAGO, IL, 60606-6473, US

(73) Assignee: Siemens Aktiengesellschaft

(21) Appl. No.: 10/340,990

(22) Filed: Jan. 13, 2003

(30) Foreign Application Priority Data
Jan. 17, 2002 (DE) 10201644.5

(51) Int. Cl. A61B 5/05

(52) U.S. Cl. 600/407

(57) ABSTRACT

In a registration procedure in projective intra-operative 3D imaging for imaging a 3D patient coordinate system onto a 3D image coordinate system using marker points secured to the patient with fixed positions relative to the body part to be displayed the position of which is acquired by the instruments to be mixed into the image, the marker points are at least partially arranged outside the reconstructable 3D volume, and the markers are acquired in at least two 2D projection images from which the 3D image is calculated and are back-projected using the projection matrices that are calculated for the respective 2D projection images, and which were determined for the reconstruction of the 3D volume set, and are brought into relationship with the marker coordinates in the patient coordinate system.

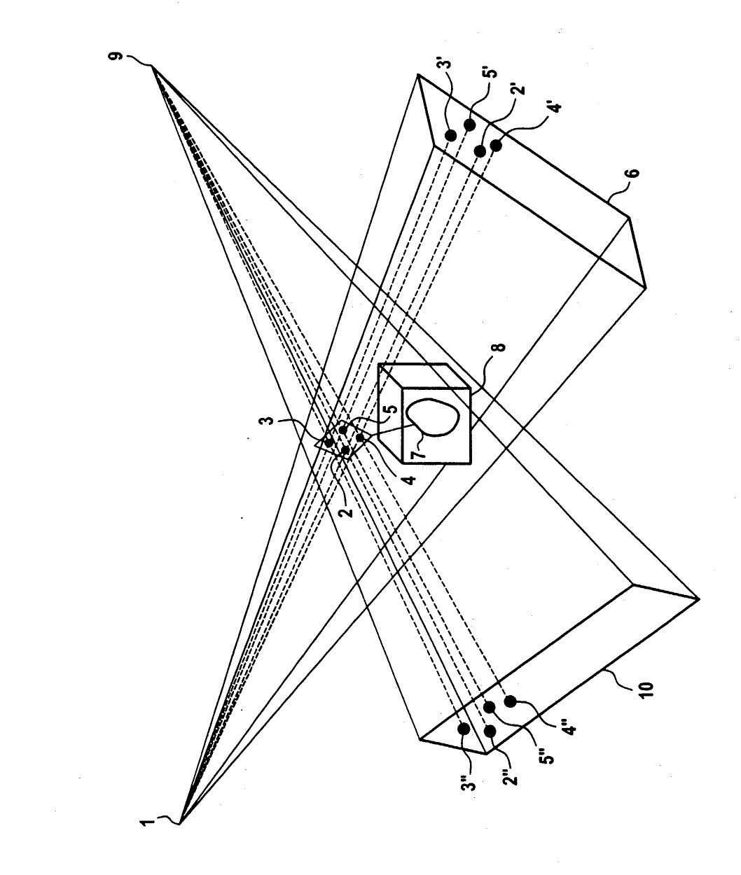

### BACKGROUND OF THE INVENTION

1. Field of the Invention

The present invention is directed to a registration procedure in projective intraoperative 3D imaging for imaging a 3D patient coordinate system onto a 3D image coordinate system of the type wherein marker points are secured to the patient at fixed positions relative to the body part to be displayed, the position of which is acquired by the instruments to be mixed into the image.

2. Description of the Prior Art

The employment of navigation systems in surgical operations requires a registration (coordinate transformation) between the image coordinates, the instruments and the patient.

This is conventionally accomplished by means of the identification of anatomical or artificial landmarks at the patient that are identified in the image dataset as well as directly at the patient via a navigation pointer, and the position of which in the respective coordinate system is determined. The positions of these point pairs enable the determination of the transformation between the various coordinate systems.

The spatial relationship between the intra-operative imaging modality and the reconstructed 3D data cube is defined by means of a markerless registration in a calibration procedure. For this, the position of the imaging apparatus in space must be determined by a navigation system. A transformation between the patient and the images is possible since the positions of the instruments are likewise known in the coordinate system of the navigation system.

In a form of imaging registration that is simpler and particularly frequently employed, marker points are applied in the volume to be reconstructed, these marker points being visible in the image in the intra-operative imaging. The required transformations can be calculated from the positions of the marker points in the image and the corresponding positions of the real markers at the patient.

In current methods with such marker registrations, all of the marker points must be contained in the reconstructed image, which is hardly possible given small volumes, as in the case of current 3D C-arm devices wherein the reconstructed image volume amounts to 12 cm³. Moreover, the attachment of the markers within the small, reconstructable 3D volume, which, of, course, represents the actual operation area, is often not possible for space reasons or because it is disruptive in the operation.

### SUMMARY OF THE INVENTION

An object of the present invention is to provide registration procedure in projective intra-operative 3D imaging with marker points secured to the patient wherein the marker points can be arranged more simply and at positions that are not disruptive.

This object is inventively achieved in a method of the above type wherein the marker points are at least partially arranged outside the reconstructable 3D volume, and wherein the markers are acquired in at least two 2D projection images from which the 3D image is calculated and are back-projected using the projection matrices that are calculated for the respective 2D projection images and which were determined for the reconstruction of the 3D volume set and are brought into relationship with the marker coordinates in the patient coordinate system.

The invention is based on the perception that the 2D projection images cover a much larger area than the 3D volume ultimately calculated from them, so that marker points outside of the calculated 3D volume are also visible in the 2D projection images. The corresponding markers can be back-projected from the individual projection images into the 3D space from the perception of the individual projection geometries (projection matrices). The position of the markers in the 3D space thus is obtained from the intersections of the corresponding straight lines without the markers having to be explicitly contained in the reconstructed volume.

In an embodiment of the invention the markers are also partially arranged outside the body of the patient. Of course, a fixed position of the markers relative to the body part to be displayed in the reconstructed 3D volume must continue to be established. For an intervention at the spinal column, for example, this can be achieved by markers lying outside the body at a location of the spinal column relatively far away from the intervention area, being rigidly connected to individual vertebrae via connecting pins. Even though the markers—at least in part—are not visible at all in the 3D image, a navigation of the instruments mixed into the 3D image can be implemented.

### DESCRIPTION OF THE DRAWING

The single FIGURE schematically illustrates the inventive registration procedure in projective intra-operative 3D imaging.

### DESCRIPTION OF THE PREFERRED EMBODIMENTS

The FIGURE shows a schematic illustration of a test subject with four markers attached thereto and the course of the projection cones of the markers in the production of two 2D projection exposures from different angles of view, for example given employment of a C-arm X-ray apparatus.

The arrangement of the X-ray apparatus in position 1 images the four markers 2 through 5 in the 2D projection image 6 as marker points 2′, 3′, 4′ and 5′, this projection image 6 also containing the subject 7 of actual interest, for example, the subject 7 to be operated upon, in a reconstructable 3D volume 8. In the same way, the markers 2 through 5 arranged outside the reconstructable 3D volume 8 are imaged from the imaging positions 9 into the positions 2″ through 5″ ins a second 2D image 10.

The markers from the individual projection images can be back-projected into the 3D space by means of the back-projection of the marker points into the 3D volume with known projection matrices. The position of the markers in the 3D space thus is obtained from the intersections of the corresponding straight lines (shown with broken lines) without the markers having to be explicitly contained in the reconstructed volume 8. In practice this means that not all markers need to lie outside the reconstructed volume 8, but at least one should have such an offset position.

Although modifications and changes may be suggested by those skilled in the art, it is the intention of the inventor to embody within the patent warranted hereon all changes and modifications as reasonably and properly come within the scope of his contribution to the art.

I claim as my invention:

1. A registration method in protective intra-operative 3D imaging comprising the steps of:
securing a plurality of marker points at respective fixed positions relative to a body part of a patient of which a 3D volume is to be displayed as a 3D image, and arranging said markers at least partially outside of said 3D volume, said patient having a 3D patient coordinate system associated therewith and said 3D image having a 3D image coordinate system associated therewith;
acquiring at least two 2D projection images each contains said markers and said 3D volume;
calculating said 3D image in said 3D image coordinate system by back-projection using respective projection matrices calculated for said at least two 2D projection images; and
imaging said 3D patient coordinate system into said 3D image coordinate system in said 3D image dependent on coordinates of said markers in said back-projection.

2. A method as claimed in claim 1 comprising arranging said markers at least partially outside of the body of said patient.
